(19) United States
(12) Patent Application Publication
(10) Pub. No.: US 2006/0282115 A1
(43) Pub. Date: Dec. 14, 2006

(54) Thin film vessel occlusion device

(76) Inventors: Robert M. Abrams, Los Gatos, CA (US); Hoa Vinh Vo, San Jose, CA (US); Robert Z. Obara, Fremont, CA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/148,803

(22) Filed: Jun. 9, 2005

(51) Int. Cl. A61M 29/00

(52) U.S. Cl. 606/200

(57) ABSTRACT

A body conduit occlusion device is provided that includes a metal thin film restriction member attached to a shaft. The restriction member is moveable between a collapsed configuration and a radially expanded configuration.

### BACKGROUND

Body vessels and conduits, for example coronary arteries, the carotid artery, and lumens of the biliary tree, are frequently treated from within using catheters having treatment devices for treating conditions or affected areas at locations within the vessels. Treatment device examples include angioplasty balloons, stents and associated stent delivery catheters, drug delivery catheters, atherectomy devices, and devices for crushing or dissolving blockages in the biliary tree. When using these and other devices, it may be desirable to position and expand an occlusion device such as an inflatable distal occlusion balloon in proximity to the device. In coronary artery applications, the occlusion device can be disposed distally and downstream of the more proximal treatment apparatus such as a rotatable atherectomy burr or an angioplasty balloon. In this application, the occlusion device is a distal occlusion device. A distal occlusion device may also be placed downstream of a stent and associated with a stent delivery catheter while the stent is being expanded against the vessel wall.

Distal occlusion devices may also be used to provide a quiescent region of a body vessel where treatment can occur. In one example, an artery may be blocked off from blood flow to allow treating a stenosed region vessel wall with an agent to inhibit restenosis. In another example, a stone may be isolated between a distal and a proximal occlusion balloon, with the space being filled with a chemical to dissolve the stone. In many of these applications, the vessel region proximal of the distal occlusion device is aspirated through a catheter lumen to remove byproducts prior to deflating or removing the distal occlusion device.

### SUMMARY

The present invention provides occlusion devices for restricting fluid flow in body conduits and vessels. The occlusion devices provide distal or proximal restriction or occlusion, depending on their position with respect to a procedure site. The devices include expandable distal portions and an elongate shaft. The occlusion devices allow other devices to be advanced over and retracted from the occlusion device shaft while the occlusion devices restrict fluid flow through the conduit or vessel.

One device includes an elongate tubular shaft having a frustoconical shaped restriction member disposed near the distal end. The restriction member is expandable and contractible to provide varying degrees of fluid flow restriction or complete vessel occlusion. In some embodiments, the restriction members are made of a single layer non-porous thin metal film to provide a low profile and higher strength than polymer occlusion devices.

In one embodiment, the restriction member is attached to the shaft proximally of the distal end of the shaft with the distal major opening facing the distal end of the shaft. In some embodiments, the restriction member is not detachable from the shaft. The restriction member is impervious to fluid flow. In some embodiments, the restriction member a single layer nitinol thin film.

Some embodiments have a restriction member with a plurality of overlapping segments. Other embodiments include a sheath sized and configured to slide over the shaft and restriction member in the collapsed configuration. The restriction member can have one or more fenestrations. Additional embodiment include a second restriction member without fenestrations attached to the shaft distal of the restriction member with fenestrations. The two restriction members have different mechanisms of moving from a collapsed configuration to an expanded configuration.

Another embodiment of vessel occlusion device includes a tubular shaft and a collapsible metal thin film restriction member attached to the distal end of the shaft such that the distal major opening of the restriction member extends beyond the distal end of the shaft. The device also includes an actuator disposed within the tubular shaft to move the restriction member between the collapsed and radially expanded configurations.

Some embodiments include a tether releasably attached to the restriction member and extending proximally through the shaft. The tether maintains the restriction member in the collapsed configuration and when released, allows the restriction member to expand to the frustoconical configuration. In other embodiments, a push rod is slidably disposed within the tubular shaft, the push rod having an outer diameter greater than an inner diameter of the restriction member in the collapsed configuration.

### BRIEF DESCRIPTION OF THE DRAWINGS

FIG. 1 illustrates a representative occlusion device according to the invention in an expanded configuration within a vessel.

FIG. 2 shows the occlusion device of FIG. 1 in a collapsed configuration.

FIG. 3 illustrates a occlusion device according to another embodiment of the invention in a collapsed configuration within a vessel.

FIG. 4 illustrates a occlusion device with a push rod for actuating the occlusion member.

FIG. 5 illustrates a occlusion device with a distally mounted fenestrated occlusion member.

FIG. 6 illustrates a occlusion device with two occlusion members; one with fenestrations and one without.

FIGS. 7A and 7B illustrate a occlusion device with overlapping segments in a partially expanded and fully expanded configuration, respectively.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. “Nitinol” or “TiNi” refers to an alloy containing titanium and nickel, typically each between 45-55 atom percent, and optionally, other metals, such as chromium in relatively minor amounts. “Sputtered alloy” refers to an alloy formed by sputter depositing a target-material alloy on a substrate, such as a mandrel.

As used in this specification and the appended claims, “restriction” refers to reducing the volume of a vessel or reducing the amount of fluid flowing through a vessel. The amount of restriction is variable between a slight reduction if fluid flow and complete blockage of a vessel. “Occlusion” refers to a substantially complete blockage of a vessel. The terms are used interchangeably to denote embodiments in which the device is adjustable between a configuration providing slight reduction in fluid flow (restriction) to a configuration providing substantially complete blockage (occlusion) of a vessel.

The following description should be read with reference to the drawings wherein like reference numerals indicate like elements throughout the several views. The drawings, which are not to scale, depict illustrative embodiments of the claimed invention.

A restriction device with a proximal end outside diameter approximately the same as the shaft outside diameter at its midpoint longitudinally can provide an elongate shaft that can be used for advancing a second medical device over the elongate shaft. The shaft can thus be used in ways similar to a guide wire. In one use, the shaft can be used to guide a therapeutic device such as an atherectomy catheter, an angioplasty catheter, or a stent delivery catheter over the shaft. In another use, the shaft can be used to guide a diagnostic device such as an angiography catheter over its length. “Over the wire” catheters can be guided to a target site, having a shaft disposed within most of their length. Single operator exchange catheters can be guided to a target site, having the elongate shaft disposed primarily within a distal region of the device. For such uses, it is preferred that the shaft have an outside diameter of about 0.010 inches to about 0.018 inches.

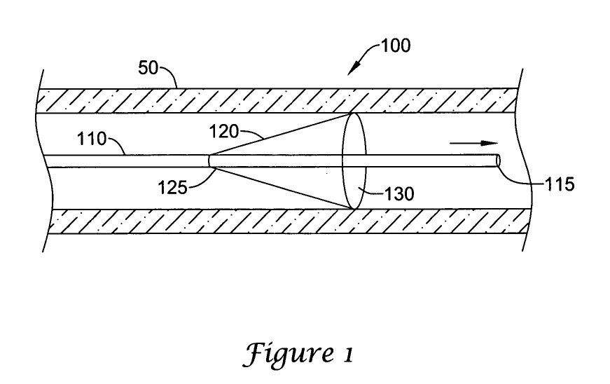

FIG. 1 illustrates a restriction device 100 in an expanded orientation within a vessel 50. The restriction device 100 includes an elongate tubular shaft 110 and a distally disposed restriction member 120. The shaft 110 can be hollow or solid. A hollow shaft 110 can include a single lumen extending from a proximal end to the distal end. In another embodiment the shaft 110 is a multi-lumen shaft.

Restriction member 120 is formed from a thin film that can be actuated between a collapsed and an expanded configuration. The thin film can be formed of a metal with shape memory and/or super elastic properties. One such material is nitinol. The thin film is formed into a frustoconical shape and disposed around an elongate shaft 110. In some embodiments the elongate shaft 110 is a catheter containing at least one lumen. The shaft 110 with attached restriction member 120 can be threaded over a guidewire to the desired location within a vessel. In other embodiments, the elongate shaft 110 is a solid guidewire. The guidewire shaft 110 with attached restriction member 120 can be extruded from a catheter at the desired location within the vessel.

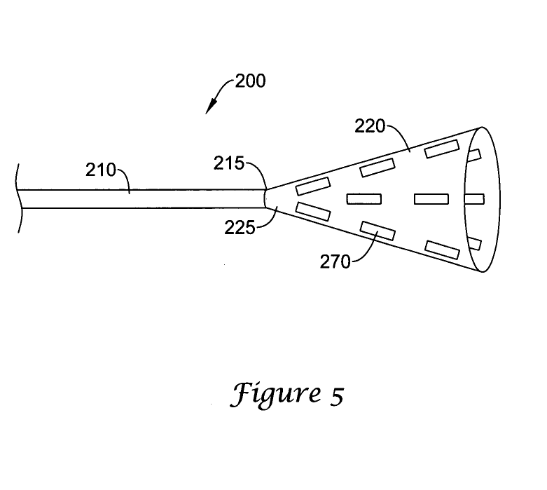

The restriction member 120 has a proximal region 125 attached to the shaft 110 and a mouth 130 that faces the distal end 115 of the shaft 110. In some embodiments, the restriction member 120 is attached proximally of the distal end 115 of the shaft 110, as shown in FIG. 1. In another embodiment, shown in FIG. 5, the proximal region 225 of the restriction member 220 is attached at the distal end 215 of the shaft 210 in such a way that the restriction member 220 extends beyond the distal end 215 of shaft 210. In some embodiments, the restriction member 120, 220 is not detachable from the shaft 110, 210.

The elongate shaft 110 can be made of a material such as stainless steel hypotubing or other materials well known to those skilled in the art such as a relatively stiff polymer or a nickel titanium alloy. The restriction member 120 can be formed by sputtering nitinol over a form, such as a mandrel. The mandrel shape is selected to provide the desired shape of restriction member, such as a frustoconical or cone shape.

One method of sputtering the nitinol onto a mandrel includes the steps of placing in a magnetron sputtering device, a mandrel having an exposed, etchable outer layer that corresponds to the open, interior volume of the device to be formed, providing the sputtering apparatus with a TiNi alloy target composed of between 45-55% each of titanium and nickel, and sputter depositing material from the target adjacent said mandrel under low-pressure, low-oxygen conditions. During the deposition, the mandrel is moved relative to said target to achieve substantially uniform sputter deposition over the entire exposed surface of the mandrel, and the deposition is continued until a desired sputtered film thickness from 0.5 microns to 35 μm is formed on the mandrel.

Following sputter deposition, the thin film on the mandrel is heated under annealing conditions. The thin-film device is then released from the mandrel, typically by exposing the mandrel and deposited thin film to an etchant, under conditions effective to dissolve the outer layer of the mandrel. The mandrel's outer layer may be a separate coating formed on the mandrel surface, or the surface of the mandrel itself. The mandrel may be coated with a smooth surface such as polyimide before sputtering to ensure a continuous layer of deposited material. When holes, slots, or fenestrations 270 are desired, a further etching process is performed on the sputtered device.

In some embodiments the restriction member 120 has a thickness of 2.0-50 microns. In other embodiments, the restriction member 120 has a thickness of 5 microns. Shaft 110 and the restriction member 120 in the collapsed configuration can have a diameter that is in the range of about 4F (French) to about 9F.

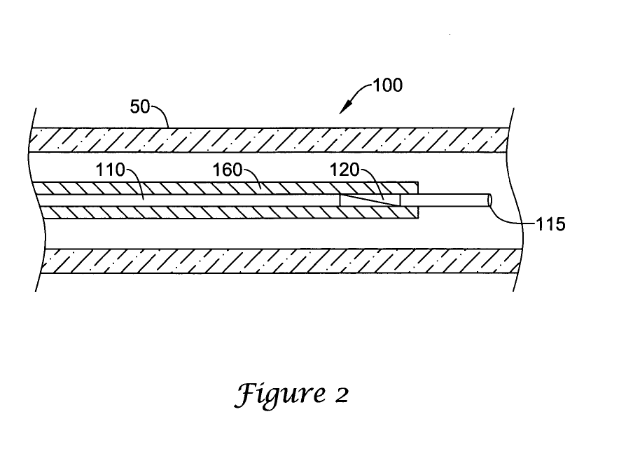

The restriction member 120 expands from a collapsed configuration, shown in FIG. 2, to an expanded configuration shown in FIG. 1. In the embodiment shown in FIG. 2, the restriction member 120 is folded against the shaft 110 in the collapsed configuration. In embodiments in which the restriction member 120 made of a self-expanding material such as a shape memory metal, a sheath 160 can be disposed over shaft 110 and collapsed restriction member 120 during delivery. See FIG. 2. Once the shaft 110 and restriction member 120 are in the desired location, the sheath 160 is withdrawn proximally, allowing self-expanding restriction member 120 to expand into an expanded, restricting configuration. After the medical procedure is complete, the sheath 160 can be used to contract the expanded restriction member 120 into a collapsed configuration for withdrawal.

In other embodiments, the restriction member 120 is made of a shape memory metal or other material that expands and contracts under specific conditions such as temperature or electrical current. In such embodiments, the restriction member 120 is folded or otherwise collapsed around the shaft 110 for delivery. Once at the desired location, the restriction member 120 is actuated to its expanded configuration.

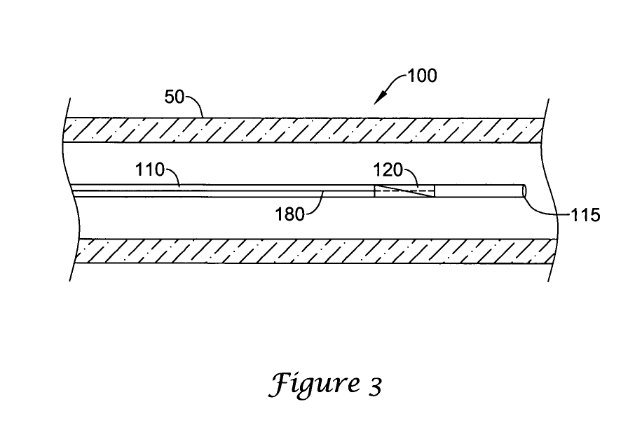

In a further embodiment, the restriction device 100 includes an actuator 180 that facilitates movement of the restriction member 120 from the collapsed to the expanded configuration. See FIG. 3. The actuator can be, for example, a push rod, one or more struts, a tether, or any other actuating member that functions to expand a collapsed restriction member 120.

In the embodiment shown in FIG. 3, the actuator is a tether 180 is releasably attached to the restriction member 120. The tether 180 extends proximally through the shaft 110. In some embodiments, the tether 180 extends through the shaft 110 to the proximal end of the shaft 110 where it is manipulated by the operator. The tether 180 maintains the restriction member 120 in the collapsed configuration. When the tether 180 is released or detached from the restriction member 120, the restriction member 120 expands to the expanded frustoconical configuration. The tether 180 can be made of any suitable material, such as wire or filament.

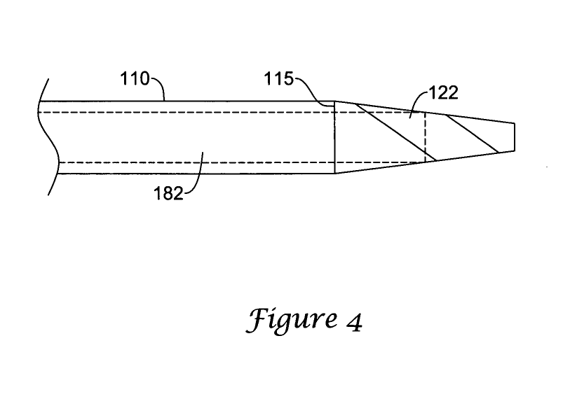

In the embodiment shown in FIG. 4, the restriction member 122 is attached to the distal end 115 of the tubular shaft 110 such that the collapsed restriction member 122 extends beyond the distal end 115. In the collapsed state, the restriction member 122 has an inner diameter less than that of the shaft 110. A push rod 182 is slidably disposed within the shaft 110. The push rod 182 is advanced distally into the collapsed restriction member 122, pushing the restriction member 122 into the expanded configuration.

In the embodiment shown in FIG. 5, the restriction device 200 includes a thin film metal restriction member 210 with one or more fenestrations 270. The fenestrations 270 allow some fluid to pass through the restriction member, thereby modifying the percent occlusion achieved by the device. In some embodiments, the fenestrations 270 are sized such that the restriction device 200 acts as a filter, trapping embolic material while allowing a reduced volume of fluid flow through the device 200.

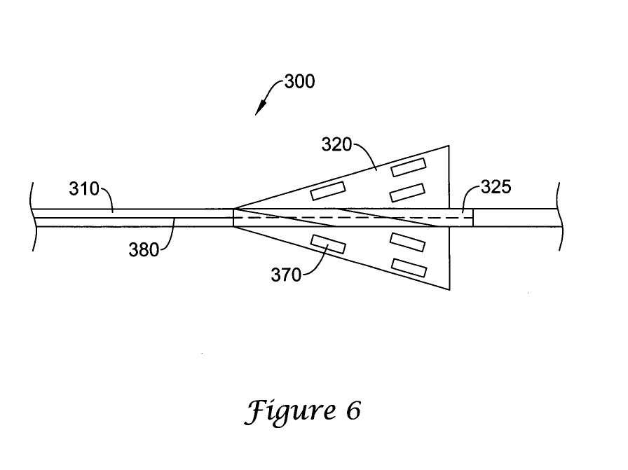

In another embodiment, shown in FIG. 6, the restriction device 300 includes two restriction members, an outer restriction member 320 with fenestrations 370 and an inner restriction member 325 without fenestrations. FIG. 6 shows the outer restriction member 320 in an expanded configuration and the inner restriction member 325 in a collapsed configuration. Expanding the outer restriction member 320 with fenestrations 370 allows some fluid to pass through, resulting in less than complete occlusion. When a further reduction in fluid flow or complete occlusion is desired, the inner restriction member 325 is also expanded, thereby at least partially blocking the fenestrations 370. Separate actuation of the outer restriction member 320 and inner restriction member 325 may be achieved by using a tether 380 to maintain the inner restriction member 325 in a collapsed configuration while a sheath (not shown) is retracted proximally, allowing the outer restriction member 320 to expand.

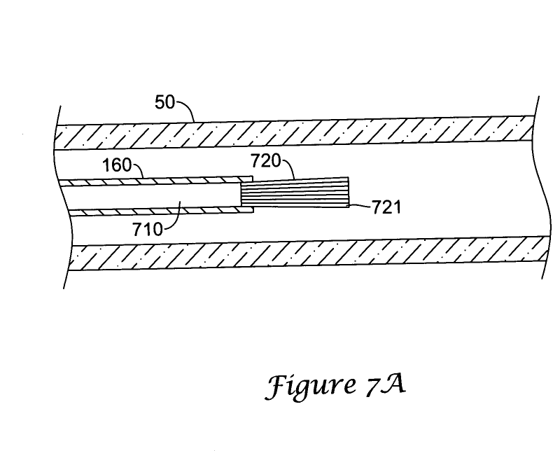

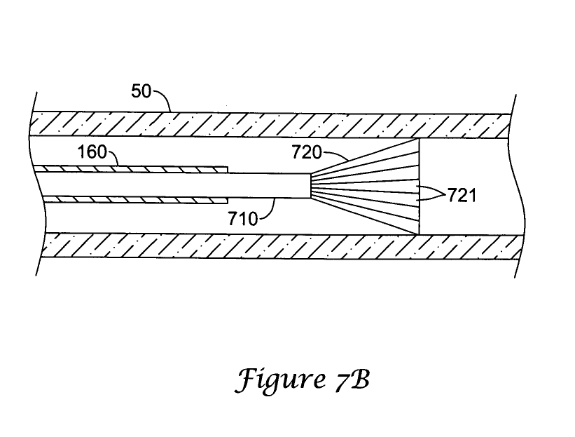

In a further embodiment, the restriction member 720 is made of a plurality of overlapping segments 721. See FIGS. 7A and 7B. The segments 721 overlap when the restriction member 720 is in the collapsed configuration within a sheath 160 (FIG. 7B). As the sheath 160 is withdrawn proximally, the segments 721 slide away from each other, expanding to form the restriction member 720 (FIG. 7B). The degree to which the restriction member 720 is expanded can be varied by adjusting the position of the sheath 160. The farther proximal the sheath 160 is moved, the greater degree of expansion of the restriction member 720.

In use, the restriction device 100 with the restriction member 120 in a collapsed configuration is advanced to a location either proximal or distal of a target site in a body conduit such as a vessel. The placement of the restriction device depends on whether proximal or distal occlusion is desired. The restriction member 120 is actuated from the collapsed configuration to the expanded configuration to occlude the vessel. With the vessel occluded, a catheter or other medical device can be used to treat the target site. In some applications, this may include advancing a catheter or other medical device over or through the shaft 110. Upon completion of the medical procedure, a sheath 160 is advanced over the shaft 110 to collapse the restriction member 120 and restore fluid flow through the vessel. The shaft 110 with collapsed restriction member 120 is withdrawn proximally within the sheath 160.

It will be understood that this disclosure is, in many respects, only illustrative. Changes may be made in details, particularly in matters of shape, size, and arrangement of parts without exceeding the scope of the invention. The invention's scope is, of course, defined in the language in which the appended claims are expressed.

What is claimed is:

1. A vessel occlusion device comprising:
an elongate shaft having a proximal region, a distal region, and a distal end; and
a single layer non-porous metal thin film restriction member having a proximal end and a distal major opening, the proximal end of the restriction member attached to the shaft proximally of the distal end with the distal major opening facing the distal end of the shaft; the restriction member moveable between a collapsed configuration and a radially expanded frustoconical configuration, wherein the single layer restriction member is impervious to fluid flow.

2. The vessel occlusion device of claim 1, wherein the restriction member is a single layer thin film made of a super elastic metal.

3. The vessel occlusion device of claim 1, wherein the restriction member is a single layer thin film made of a shape memory metal.

4. The vessel occlusion device of claim 1, wherein the restriction member is a single layer nitinol thin film.

5. The vessel occlusion device of claim 1, wherein the restriction member is not detachable from the shaft.

6. The vessel occlusion device of claim 1, further comprising a tether releasably attached to the restriction member and extending proximally through the shaft, wherein the tether maintains the restriction member in the collapsed configuration and when released, allows the restriction member to expand to the frustoconical configuration.

7. The vessel occlusion device of claim 1, wherein the restriction member includes a plurality of overlapping segments.

8. The vessel occlusion device of claim 1, further comprising a sheath sized and configured to slide over the shaft and restriction member in the collapsed configuration.

9. The vessel occlusion device of claim 1, wherein the restriction member includes one or more fenestrations.

10. The vessel occlusion device of claim 9, further comprising a second restriction member without fenestrations attached to the shaft distal of the restriction member with fenestrations.

11. The vessel occlusion device of claim 10, wherein the restriction member with fenestrations and the second restriction member without fenestrations have different mechanisms of moving from a collapsed configuration to an expanded configuration.

12. A vessel occlusion device comprising:
an elongate tubular shaft having a proximal end and a distal end and a lumen extending therethrough;
a collapsible metal thin film restriction member having a proximal end and a distal major opening, the proximal end attached to the distal end of the shaft such that the distal major opening of the restriction member extends beyond the distal end of the shaft, the collapsible metal thin film restriction member moving between a collapsed configuration and a radially expanded configuration; and
an actuator disposed within the tubular shaft, the actuator moving the restriction member between the collapsed and radially expanded configurations.

13. The vessel occlusion device of claim 12, wherein the actuator is a tether attached to the restriction member and extending proximally through the tubular shaft; wherein the tether maintains the restriction member in the collapsed configuration; wherein releasing the tether results in expansion of the restriction member to the expanded configuration.

14. The vessel occlusion device of claim 12, wherein the actuator is a push rod slidably disposed within the tubular shaft, the push rod having an outer diameter greater than an inner diameter of the restriction member in the collapsed configuration.

15. The vessel occlusion device of claim 12, wherein the restriction member includes a plurality of overlapping segments.

16. The vessel occlusion device of claim 12, wherein the collapsible metal thin film restriction member is a single layer that is substantially impervious to fluid flow.

17. The vessel occlusion device of claim 12, wherein the restriction member includes one or more fenestrations.

18. The vessel occlusion device of claim 17, further comprising a second restriction member attached to the shaft distal of the restriction member with fenestrations.

19. The vessel occlusion device of claim 12, wherein the restriction member is a single layer thin film made of a super elastic metal.

20. The vessel occlusion device of claim 12, wherein the restriction member is a single layer thin film made of a shape memory metal.

21. The vessel occlusion device of claim 12, wherein the restriction member a single layer nitinol thin film.

22. The vessel occlusion device of claim 12, wherein the restriction member is not detachable from the shaft.

23. A method of treating a target site in a body conduit while temporarily occluding the conduit, the method comprising:
providing a restriction device having a single layer non-porous metal thin film restriction member in a collapsed configuration on a shaft, the restriction member actuatable between a collapsed configuration and a radially expanded configuration;
advancing the restriction device to a desired location either proximal or distal of the target site in the body conduit;
actuating the restriction member from the collapsed configuration to the expanded configuration to occlude the body conduit;
treating the target site;
collapsing the restriction member; and
withdrawing the restriction device from the body conduit.

24. The method of treating a target site in a body conduit of claim 23, wherein the restriction member is a single layer thin film made of a super elastic metal.

25. The method of treating a target site in a body conduit of claim 23, wherein the restriction member is a single layer thin film made of a shape memory metal.

26. The method of treating a target site in a body conduit of claim 23, wherein the restriction member a single layer nitinol thin film.
